(19) United States
(12) Patent Application Publication
(10) Pub. No.: US 2008/0293585 A1
(43) Pub. Date: Nov. 27, 2008

(54) 5'/3' Ratioing Procedure for Detection of Gene Rearrangements

(75) Inventors: Gabor Szabo, Debrecen (HU); Gyorgy Lustyik, Pecs (HU)

Correspondence Address: DAVIS WRIGHT TREMAINE LLP/Los Angeles, 865 FIGUEROA STREET, SUITE 2400, LOS ANGELES, CA, 90017-2566, US

(73) Assignee: Cedars-Sinai Medical Center, Los Angeles, CA (US)

(21) Appl. No.: 11/910,077

(22) PCT Filed: Apr. 17, 2006
(86) PCT No.: PCT/US06/14392

§ 371 Date: Sep. 28, 2007

Related U.S. Application Data
(60) Provisional application No. 60/671,949, filed on Apr. 15, 2005.

(51) Int. Cl. C12Q 1/68; C40B 30/04

(52) U.S. Cl. 506/9; 435/6

(57) ABSTRACT

The present invention relates to methods and kits useful for the detection of gene rearrangements and the diagnosis of a propensity to develop a disease condition caused by the gene rearrangements, wherein two PCR products are prepared from the 5′ side and from the 3′ side of a putative breakpoint of the gene of interest, and the ratio of the two products are measured.

### GOVERNMENT RIGHTS

The U.S. Government has certain rights in this invention pursuant to Grant No. R43CA96379-01 awarded by the National Cancer Institute.

### FIELD OF THE INVENTION

The invention relates to methods and kits for the detection of gene rearrangements.

### BACKGROUND OF THE INVENTION

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Accurate diagnostic tools specific to cancer and other illnesses are important for high quality patient care. Early detection is critical for treating many such conditions, but this can not be accomplished without a practical means for assessing an individual's propensity to develop the disease. This is particularly important for the treatment of diseases, such as various forms of cancer, in which the patient typically does not present symptoms until the disease has progressed substantially.

Chromosomal rearrangements, also called translocations, are a hallmark of many types of cancer; a greater number of rearrangements generally indicates a higher probability of developing cancer. For example, in the case of acute leukemia, gene rearrangements often occur within the mixed-lineage leukemia gene (MLL), which is the human homologue of the Drosophila gene trithorax, and is involved in pattern development during embryogenesis. The translocation breakpoints within the MLL gene almost always fall within a limited region of 8.3 kilobases, referred to as the MLL breakpoint cluster region (bcr). MLL translocations result in the generation of fusion proteins that retain the MLL amino-terminus. Transformation of cells by rearranged forms of MLL, including in-frame fusion proteins, partial tandem duplications, and amplification of MLL through up regulation of Hox gene and cofactor expression often blocks hematopoietic differentiation. MLL rearrangements are associated with aggressive, acute leukemias in infants, children, and adults, and are found in leukemias with both lymphoid and myeloid phenotypes.

Based on the relationship between chromosomal rearrangements and neoplastic disease, there is a significant need in the art to have the capability to detect gene rearrangements such as those associated with MLL. Such detection systems can identify gene rearrangements that often pre-date the onset of and/or provide treatment options early in the progression of cancer.

### SUMMARY OF THE INVENTION

The following embodiments and aspects thereof are described and illustrated in conjunction with methods and kits are meant to be exemplary and illustrative, not limiting in scope.

The present invention relates to methods and kits useful for the detection of gene rearrangements and diagnosing a propensity to develop a disease condition due to gene rearrangements.

A method of detecting a gene rearrangement comprises providing a quantity of a polynucleotide, said polynucleotide including a region of interest, cleaving said polynucleotide to create polynucleotide fragments (e.g., using a restriction enzyme such as BamHI), a portion of which are capable of binding to a probe, combining a quantity of the probe with said polynucleotide fragments to hybridize at least a portion of the polynucleotide fragments, removing a substantial quantity of the hybridized polynucleotide fragments to create a remaining sample and a removed sample and quantitating the amount of a first section of the region of interest and a second section of the region of interest in the remaining sample or the removed sample to detect the gene rearrangement. In one embodiment, the first section may be on the 5′ side of a putative breakpoint and the second section may be on the 3′ side of a putative breakpoint.

In one embodiment, quantitating the amount of the first section and the second section comprises introducing to the remaining sample or the removed sample a quantity of a first primer pair targeting the first section and a quantity of a second primer pair targeting the second section, amplifying the first and second sections to generate a first product and a second product, and quantitating the amount of the first product and the second product to detect the gene rearrangement. The first primer pair and the second primer pair may be introduced at the same time or separately. Likewise, amplifying the first and the second sections to generate the first and the second products may be performed together or separately.

Quantitating the amount of the first section and the second section may also be performed by a variety of techniques; for example, quantitative polymerase chain reaction (qPCR), gel electrophoresis, using microbeads in combination with flow cytometry, using gene-chip analysis, and using microarray analysis.

In one embodiment, the polynucleotide is genomic DNA. In another embodiment the polynucleotide is cDNA. In another embodiment, the polynucleotide is mRNA.

In one embodiment, the probe may be immobilized on a solid matrix. In another embodiment, the probe is one that has an affinity tag (e.g., biotin).

In one embodiment, removing a substantial quantity of the hybridized polynucleotide fragments to create the remaining sample and the removed sample comprises providing a solid matrix comprising avidin or similar biotin-binding proteins and contacting biotinylated polynucleotide fragments to the avidin, wherein the biotinylated polynucleotide fragments bind to the avidin and are removed from the sample to create the remaining sample and the removed sample.

In another embodiment, the sample comprising the polynucleotide fragments may be contacted with biotinylated probes that have been immobilized on a solid matrix comprising avidin or similar biotin-binding proteins. In this embodiment, the immobilized probes will hybridize with the target fragments and separation of the solid matrix (with the immobilized probes) and the sample results in removal of the target polynucleotide fragments from the sample.

In one embodiment, the region of interest is the mixed-lineage leukemia (MLL) breakpoint cluster region (bcr), thus the method detects gene rearrangements of the MLL gene.

In embodiments in which the probe is a probe that targets the second section of the polynucleotide, if the remaining sample comprises a larger quantity of the first section as compared to the second section, it indicates gene rearrangements, if the remaining sample comprises a substantially equal quantity of the first section and the second section, it indicates a lack of gene rearrangements and if the remaining sample lacks the first section and the second section, it indicates a lack of gene rearrangements; or if the removed sample comprises a larger quantity of the second section as compared to the first section, it indicates gene rearrangements, and if the removed sample comprises a substantially equal quantity of the first section and the second section, it indicates a lack of gene rearrangements.

In embodiments in which the probe is a probe that targets the first section of the polynucleotide, if the remaining sample comprises a larger quantity of the second section as compared to the first section, it indicates gene rearrangements, if the remaining sample comprises a substantially equal quantity of the first section and the second section, it indicates a lack of gene rearrangements, and if the remaining sample lacks the first section and the second section, it indicates a lack of gene rearrangements; or if the removed sample comprises a larger quantity of the first section as compared to the second section, it indicates gene rearrangements, and if the removed sample comprises a substantially equal quantity of the first section and the second section, it indicates a lack of gene rearrangements.

Other features and advantages of the invention will become apparent from the following detailed description, taken in conjunction with the accompanying figures, which illustrate, by way of example, various features of embodiments of the invention.

### BRIEF DESCRIPTION OF THE FIGURES

Exemplary embodiments are illustrated in the referenced figures. It is intended that the embodiments and figures disclosed herein are to be considered illustrative rather than restrictive.

FIG. 1 depicts a method of detecting rearranged genes in accordance with an embodiment of the present invention. FIG. 1A depicts polynucleotide fragments that were generated after cleaving a polynucleotide that contained a gene rearrangement. FIG. 1B depicts the hybridization step with a biotinylated probe selecting for 3′ fragments. FIG. 1C depicts fragments removed by the biotinylated probe. FIG. 1D depicts fragments remaining in the sample. It is noted that although the polynucleotide fragments are represented by a single line, it is not to be taken that the polynucleotide must be single stranded. In embodiments in which the polynucleotide is double stranded DNA, double stranded polynucleotide fragments are in the sample. In embodiments in which the polynucleotide is RNA, single stranded polynucleotide fragments are in the sample.

FIG. 2A depicts a theoretical gene rearrangement at a bcr close to the 3′ end of the MLL (accession number: U04737) locus modeled by EcoRV restriction enzyme digestion in accordance with an embodiment of the present invention. DNA, plasmid (pMEPMLL) or human genomic, were either digested with EcoRV before hybridization or uncleaved. A primer pair (SEQ ID NO.: 2 and SEQ ID NO.: 3) was designed for the 5′ side of the EcoRV digestion site to generate a 778 bp product (“A”). Another primer pair (SEQ ID NO.: 4 and SEQ ID NO.: 5) was designed for the 3′ side of the EcoRV digestion site to generate a 541 bp product (“B”).

FIG. 2B depicts various elements of the region of interest in accordance with the embodiments of the present invention. “A” and “B” represent the sections of the region of interest that is quantitated or amplified in accordance with various embodiments of the present invention.

FIG. 3 depicts a titration of DNA concentration in accordance with an embodiment of the present invention. The upper band is the PCR product, the lower band is the reannealed primers (therefore double stranded, i.e., binding ethidium bromide). The panel shows that the signal depends on the template concentration applied. Thus, this semiquantitative method (i.e., gel rather than qPCR, real-time or as measured in quantitative microbead assay, by FACS) provided a good estimate of template DNA. Thus, it can also be used to determine the ratio of the two PCR products to show whether a gene rearrangement is present. Lane 1: 7.4 ng; Lane 2: 7.4 ng; Lane 3: 3.7 ng; Lane 4: 1.85 ng; Lane 6: 0.925 ng; Lane 7: PCR markers—50, 150, 300, 500, 750 and 1000 bp.

FIG. 4 depicts 5′/3′ ratioing modeled by EcoRV cleaved plasmid DNA in accordance with an embodiment of the present invention. The two gels (A and B) show the same conclusion in two independent experiment models. DNA was digested with EcoRV before hybridization. As seen in Gel A, lanes 2 and 4 and in Gel B, lanes 4 and 6, hybridization removed most of or all of the template containing the “B” region (the “B” region is depicted in FIG. 2A). Therefore, only or mostly the A region was copied during the two PCR reactions (with the primers (SEQ ID NO.: 2 and SEQ ID NO.: 3) for generating the A region, and in the second reaction, with primers (SEQ ID NO.: 4 and SEQ ID NO.: 5) for generating the B region). As shown by the arrow, almost no product was produced in the PCR reaction for the B region. The small amount of the “B” product is the weak band; the smear below it was due to the reannealed primers. Gel (A): Lane 1: 150 ng of template DNA, product A; Lane 2: 150 ng of template DNA, product B; Lane 3: 90 ng of template DNA, product A; Lane 4: 90 ng of template DNA, product B; Lane 5: marker. Gel (B): Lane 1: marker; Lane 2: 250 ng of template DNA, product A; Lane 3: 250 ng of template DNA, product B; Lane 4: 350 ng of template DNA, product A; Lane 5: 350 ng of template DNA, product B.

FIG. 5 depicts 5′/3′ ratioing modeled on intact, supercoiled-plasmid DNA to demonstrate the ratio of products that may be seen when there is no rearrangement, in accordance with an embodiment of the present invention. No PCR products were detected. The visible band is the primer band. Lane 1: marker; Lane 2: 10 ng of template DNA, product A; Lane 3: 10 ng of template DNA, product B; Lane 4: 20 ng of template DNA, product A. When the DNA is intact, it is completely or almost completely removed in the hybridization and removal steps.

FIG. 6 depicts 5′/3′ ratioing modeled by EcoRV-cleaved plasmid DNA without the hybridization step. Both products A and B are equally detected. The products of the two PCR reactions were loaded on different lanes. Lane 1: marker; Lane 2: 350 ng of template DNA, product A; Lane 3: 350 ng of template DNA, product B; Lane 4: primer only.

FIG. 7 depicts 5′/3′ ratioing procedure modeled by EcoRV-cleaved human genomic DNA in accordance with an embodiment of the present invention. Lane 1: marker; Lane 2: 2 μg of template DNA, product A; Lane 3: 2 μg of template DNA, product B. The ratio of products B/A greatly decreased at 2 μg of sample DNA.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994); March, Advanced Organic Chemistry Reactions, Mechanisms and Structure 4th ed., J. Wiley & Sons (New York, N.Y. 1992); and Sambrook and Russell, Molecular Cloning: A Laboratory Manual 3rd ed., Cold Spring Harbor Laboratory Press (Cold Spring Harbor, N.Y. 2001) provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“DNA” is meant to refer to a polymeric form of deoxyribonucleotides (i.e., adenine, guanine, thymine and cytosine) in double-stranded or single-stranded form, either relaxed or supercoiled. This term refers only to the primary and secondary structure of the molecule, and does not limit it to any particular tertiary forms. Thus, this term includes single- and double-stranded DNA found, inter alia, in linear DNA molecules (e.g., restriction fragments), viruses, plasmids, and chromosomes. In discussing the structure of particular DNA molecules, sequences may be described herein according to the normal convention of giving only the sequence in the 5′ to 3′ direction along the non-transcribed strand of DNA (i.e., the strand having the sequence homologous to the mRNA). The non-transcribed strand is also referred to as the “coding strand” or the “sense strand”. The complementary DNA strand, which is used as the template to produce mRNA, is referred to as the “non-coding strand” or the “antisense” strand. The term “DNA” captures molecules that include the four bases adenine, guanine, thymine and cytosine, as well as molecules that include base analogues which are known in the art.

“Polymerase Chain Reaction” or “PCR” (U.S. Pat. No. 4,683,202) refers to a process for amplifying any desired specific nucleic acid sequence contained in a nucleic acid or mixture thereof. The process comprises treating separate complementary strands of the nucleic acid with a molar excess of two oligonucleotide primers, and extending the primers to form complementary primer extension products which act as templates for synthesizing the desired nucleic acid sequence. The steps of the reaction may be carried out stepwise or simultaneously and can be repeated as often as desired. The primers may incorporate a variety of features, including fluorescent labels, affinity tags such as biotin, avidin or streptavidin, or recognition sites for nucleases.

A “gene” or “coding sequence” or a sequence which “encodes” a particular protein is a nucleic acid molecule that is transcribed (in the case of DNA) and translated (in the case of mRNA) into a polypeptide in vitro or in vivo when placed under the control of appropriate regulatory sequences. The boundaries of the genes are determined by a start codon at the 5′ (i.e., amino) terminus and a translation stop codon at the 3′ (i.e., carboxy) terminus. A gene can include, but is not limited to cDNA from prokaryotic or eukaryotic mRNA, genomic DNA sequences from prokaryotic or eukaryotic DNA, and even synthetic DNA sequences. A transcription termination sequence will usually be located 3′ to the gene sequence.

The term “hybridization” refers to a process by which single stranded nucleic acids are allowed to interact so that complexes or hybrids are formed by molecules with sufficiently similar, complementary sequences. Double-stranded DNA may be denatured by heat or chemical means to produce single-stranded DNA that is capable of hybridization. Hybrids can be formed by DNA, RNA, or a combination including one strand of each.

The term “microbeads” refers to conventional polymeric or synthetic microbeads that may be composed of a number of substances, including polystyrene, corboxyl-styrene, or other carboxylated compounds. Antibodies can be covalently attached to microbeads for immunoassay-type studies. Alternatively, PCR products may be prepared using biotinylated and fluorescent dye-labeled primers on the two ends. Furthermore, microbeads may be used in conjunction with, for instance, quantitative PCR. Any of these methodologies may be applied alone (i.e., for titration of a single molecule) or, in part because they can be easily addressed by fluorescent dyes, in a multiplex format (i.e., using a series of microbeads resolved side-by-side in a flow cytometer).

“Biological microbeads” include fixed prokaryotic or eukaryotic cells (e.g., bacteria, yeast, etc.). These biological microbeads can be used in the same fashion as conventional polymeric or synthetic microbeads. See, e.g., Krupa et al., “Quantitative bead assay for hyaluronidase and heparinase I,” 319 Analytical Biochemistry 280-286 (2003); Yan et al., “Microsphere-based duplexed immunoassay for influenza virus typing by flow cytometry,” 284 J. Immunological Methods 27-38 (2004); Xu et al., “Multiplexed SNP genotyping using the Qbead system: a quantum dot-encoded microsphere-based assay,” Nucleic Acids Research, vol. 31, no. 8 (2003); and Kellar & Douglass, “Multiplexed microsphere-based flow cytometric immunoassays for human cytokines,” 279 J. Immunological Methods 277-285 (2003).

“Disease” or “disease condition” as used herein may include, but are in no way limited to physiological and pathological conditions, whether commonly recognized as diseases or not, that relate to or that are caused by genetic alterations. Particular conditions and disease conditions that are believed to be appropriate to diagnose in connection with various embodiments of the present invention include conditions and disease conditions related, but are in no way limited to cancer and immunological conditions (e.g., the gene rearrangements involving the T-cell receptor and immunoglobulin genes).

“Region of interest” as used herein refers to a region in a gene or a gene fragment comprising a possible location for a rearrangement or translocation.

“5′ fragment” as used herein refers to a polynucleotide fragment that is on the 5′ side of a putative breakpoint in a region of interest (e.g., a fragment on the 5′ side of a gene where a translocation and rearrangement may occur). “3′ fragment” as used herein refers to a polynucleotide fragment that is on the 3′ side of a putative breakpoint of a region of interest (e.g., a fragment on the 3′ side of a gene where a translocation and rearrangement may occur).

The inventors have developed a novel method for detecting gene rearrangements. The inventive method may be particularly useful in detecting gene rearrangements associated with various forms of cancer, although the invention may have application in detecting gene rearrangements associated with other diseases and physiologic conditions, whether or not such conditions are viewed as deleterious to one's health.

The method is based on determining the ratio of two sections, the left and the right side of a putative breakpoint in a polynucleotide where a gene rearrangement may occur. In another embodiment, the method is based on determining the ratio of polymerase chain reaction (PCR) products prepared from the left and right side of a putative breakpoint in a segment of a polynucleotide, such as DNA, where a gene rearrangement is likely to occur.

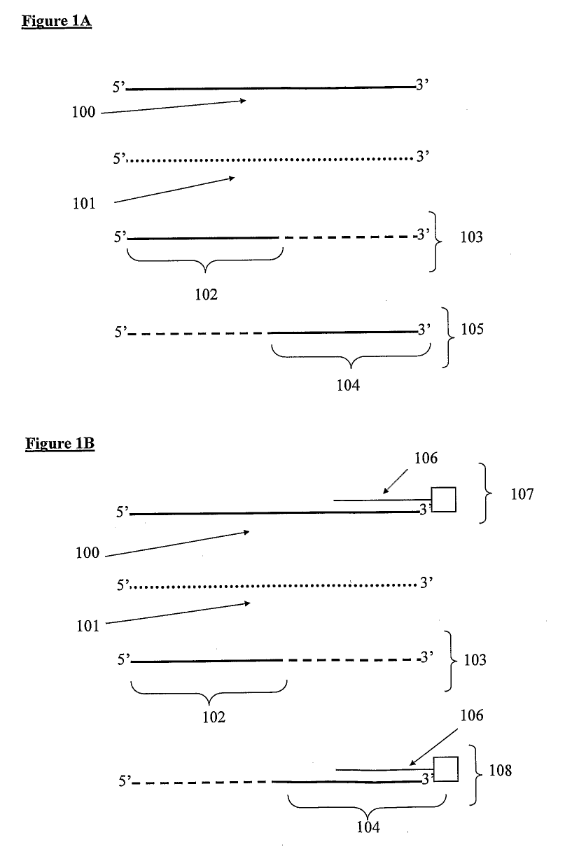

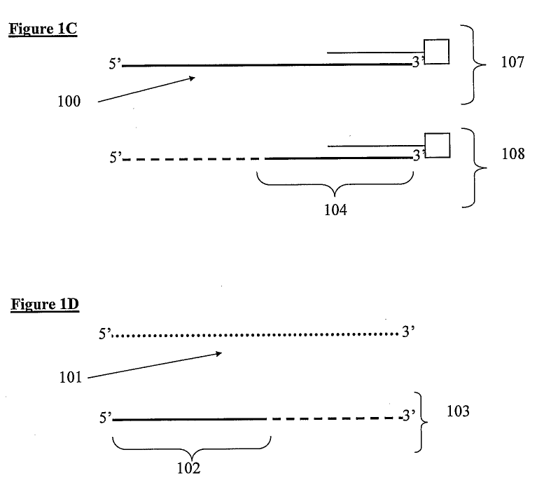

Generally referring to FIG. 1, a quantity of a polynucleotide (e.g., genomic DNA, cDNA, mRNA) is cleaved by a cleavage agent (e.g., a restriction enzyme) into multiple polynucleotide fragments 100, 101, 103, and 105. Types and relative sizes of fragments generated are not limited to those depicted in FIG. 1, as other types and sizes of fragments may be generated. Fragment 100 is an unrearranged gene or gene fragment. Fragment 101 is a polynucleotide fragment that may not be of any interest; for example, it may be a fragment of a gene that is not of any interest. Fragments 103 and 105 are rearranged genes or gene fragments. The fragments depicted in FIG. 1 illustrate an embodiment of the present invention wherein a gene rearrangement is present. In embodiments where there are no gene rearrangements, fragments 103 and 105 may not be present. The cleavage agent used will depend on the gene rearrangement to be detected. In one embodiment, the cleavage agent is one that does not cleave the polynucleotide in a region in which a gene rearrangement is likely to occur. For example, in the instance of the MLL gene, BamHI or any other enzymes delimiting a sequence which includes the breakpoint cluster region of interest can be used.

Next, a hybridization step is performed by placing a probe in contact with the polynucleotide fragments. In one embodiment, hybridization of the probe to a polynucleotide fragment may be achieved by mixing, heating and annealing the probe to the polynucleotide fragment that the probe is designed to select. The probe may select for polynucleotide fragments on the 5′ side (fragment 102) or on the 3′ side (fragment 104) of a putative breakpoint of the gene or gene fragment of interest. If the probe is one that selects for polynucleotide fragments on the 3′ side (fragment 104), the probe will hybridize with a portion of the fragment on the 3′ side. Thus, the probe will hybridize with unrearranged gene fragment 100 and the rearranged gene fragment 105. The probe may contain an affinity tag; for example, the probe may be a biotinylated probe 106.

Removal of polynucleotide fragments from the sample may be accomplished by using a probe that contains an affinity tag, for example, biotin. Other affinity tags include, but are not limited to haptens, digoxigenin, and fluorescein, which may be used for the purpose of immobilizing the probes to a solid matrix; for example, when the solid matrix carries covalently attached antibodies. The biotin-containing DNA fragments can then be bound to avidin that has been conjugated to a solid matrix, removing them from the solution phase of the sample. In an alternative embodiment, the probes may be immobilized to a solid matrix and then contacted with the sample to hybridize and remove the polynucleotide fragments that the probes are designed to remove.

The hybridized polynucleotide fragments 107 and 108 may be removed from the sample. After the removal of these polynucleotide fragments 107 and 108, the remaining sample comprises polynucleotide fragments 101 and 103. The remaining sample is quantitated for the presence of 5′ fragments (102) and 3′ fragments (104). Alternatively, the removed sample may be quantitated for the presence of 5′ fragments and 3′ fragments. As depicted in FIG. 1, the remaining sample does not comprise 3′ fragments (104). Thus when the remaining sample is quantitiated for the presence of 5′ fragments (102) and 3′ fragments (104), only 5′ fragments (102) will be detected.

Samples that do not contain gene rearrangements will not comprise fragments 103 and 105 that are present in FIG. 1. Instead “fragments” 102 and 104 have not translocated to a different gene and thus are continuous as one fragment; i.e., fragment 100. Thus, the use of the 3′ probe will hybridize and remove fragment 100. The remaining sample will not comprise 5′ fragments or 3′ fragments. Thus, quantitating the remaining sample for these fragments will not result in the detection of these fragments. Alternatively, the remaining sample may comprise 5′ fragments and 3′ fragments in a substantially equal amount due to a lack of complete removal, for example, in instances in which the probes were completely saturated. Thus, quantitating the remaining sample for these fragments will result in the detection of both fragments in trace amounts, i.e., the ratio of the two would be equal or substantially equal.

In alternative embodiments, the hybridization step may not be completely effective; for example, due to the saturation of the probes. The hybridization and removal steps may be repeated in accordance with the concentration of the polynucleotide sample. Nonetheless, in embodiments wherein the hybridization and removal steps did not remove all of the target fragments, a few scenarios may be observed. If the quantitation of the remaining sample detects substantially equal amounts of fragments 102 and 104, this indicates that no gene rearrangement is present. This is due to the fact that a small quantity of fragment 100, the unrearranged gene or gene fragment of interest may have remained in the system. Thus, both the 5′ side and the 3′ side of fragment 100 are detected in a substantially equal amount. If the probe was to hybridize and remove the polynucleotide fragments containing the 3′ fragments (e.g., 100, 105), quantitation of the remaining sample detects a large amount of 5′ fragments (102) as compared to 3′ fragments (104), this indicates that gene rearrangements are present. This is due to the fact that a small quantity of fragments 100 and/or 105 remained in the remaining sample.

In another embodiment, the removed sample may be quantitated to determine the ratio of the 5′ fragments to 3′ fragments.

There are a number of methods that may be used to quantitate the remaining sample or the removed sample, including, for example, quantitative polymerase chain reaction (qPCR), gel electrophoresis, a gene-chip or microarray analysis, and the use of microbeads in combination with flow cytometry. Quantization of the remaining sample or the removed sample can be conveniently and sensitively performed by flow-cytometry, as described and demonstrated by Pataki et al., Biological microbeads for flow-cytometric immunoassays, enzyme titrations, and quantitative PCR. Cytometry 2005 November; 68(1):45-52. Biological microbeads may be substituted for synthetic or polymeric microbeads. The PCR primers may incorporate one or more fluorescent dyes, such as 6 FAM or Cy3, to facilitate detection of the product.

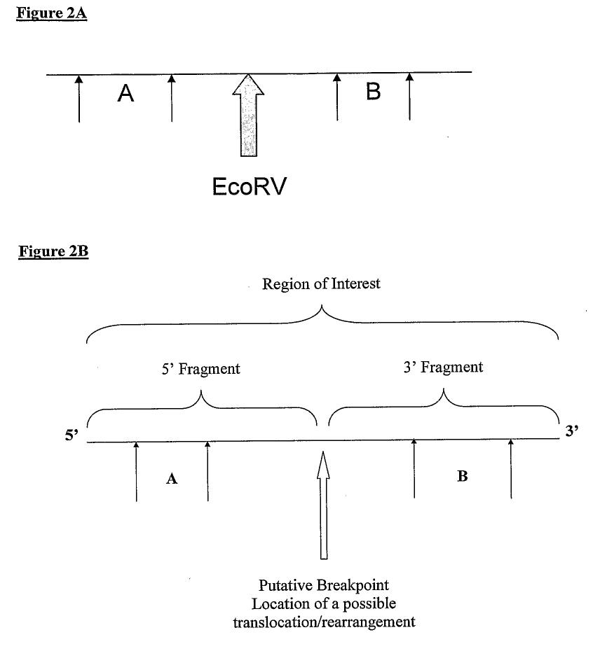

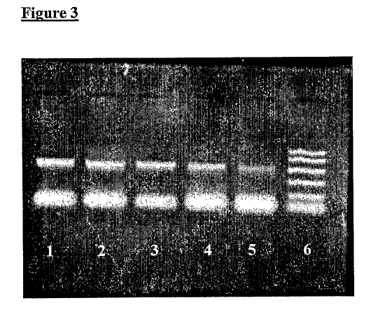

One particular method of quantitating the remaining sample may be accomplished by introducing to the remaining sample a quantity of a first primer pair that targets a first section on the polynucleotide; for example, a section on the 5′ fragment (e.g., region A on FIG. 2B). The remaining sample may also be introduced with a quantity of a second primer pair that targets a section on the polynucleotide; for example, a section on the 3′ fragment (e.g., region B on FIG. 2B). Next, PCR is performed to amplify the first section and the second section to generate a first product and a second product. The PCR may be performed as a single reaction or as separate reactions. The first product and the second product can be quantitated to detect the gene rearrangement; for example, by electrophoresis. Further, the first and the second primers may comprise a label (e.g., a fluorescent marker, an isotopic marker) to assist in the detection of the first and the second products.

In one embodiment, the first product generated by the first primer pair may be used as the probe to select and hybridize a first section on the polynucleotide; for example, to hybridize with a section on the 5′ fragment. In another embodiment, the second product generated by the second primer pair may be used as the probe to select and hybridize a second section on the polynucleotide; for example a section on the 3′ fragment.

The ratio is based on the relative amount of 5′ fragments that remain in the sample when compared with the amount of 3′ fragments that remain in the sample. A ratio different from 1:1 indicates a greater presence of the gene rearrangement. Whether the ratio is higher or lower than 1:1 will depend on whether a 3′ or a 5′ probe was used to remove the target sequences (i.e., the unrearranged sequences and either the 3′ fragments (using a 3′ probe) or the 5′ fragments using a 5′ probe). A 1:1 or substantially 1:1 ratio or the absence of 5′ segments and 3′ segments indicate a lesser presence or an absence of the gene rearrangement. Alternatively, the ratio may be based on the relative amount of the 5′ fragments that are removed from the sample when compared with the amount of 3′ fragments that remain in the sample.

In embodiments of the present invention wherein a polynucleotide associated with a cancer is being investigated with the inventive technique, these ratios may be correlated with, for example, a propensity to develop the cancer.

The method disclosed herein enables detection of gene rearrangements regardless of the identity of the heterologous gene fragment found downstream of the breakpoint following gene rearrangement. Therefore, it does not depend on additional steps necessary to identify the protein downstream of the breakpoint, a process that requires many more pairs of PCR primers and is difficult to automate.

The present invention may also use cDNA, which may result in higher sensitivity. The use of cDNA necessitates less template concentration and thus would not easily exhaust the binding capacity of the probes during the hybridization and removal of the 3′ fragments (or 5′ fragments), the non-cleaved DNA and/or the non-rearranged DNA (e.g., the use of GeneFix® tubes; available from Izotop, Inc., Hungary) as in the case of genomic DNA. In addition cDNA for a gene is a few kb long and does not contain repetitive sequences. Thus, it may be easier to devise primers.

In the case of MLL, the inventors devised a model which represents a break in the region that is frequently involved in rearrangements. For sensitivity to all rearrangements within a larger region, two distant primer pairs may be devised (e.g., about 10 kb away from each other). In such embodiments, a 10 kb DNA may be removed in the hybridization step with a probe similar to using the probe to remove a shorter DNA fragment.

The present invention is also directed to a kit for detecting gene rearrangements. The kit is useful for practicing the inventive method of detecting gene rearrangements. The kit is an assemblage of materials or components. Thus, in some embodiments the kit contains restriction enzymes, probes and/or primers as described above.

The exact nature of the components configured in the inventive kit depends on its intended purpose. For example, some embodiments are configured for the purpose of detecting gene rearrangements. In one embodiment, the kit is configured particularly for the purpose of detection in mammalian subjects. In another embodiment, the kit is configured particularly for the purpose of detection in human subjects. In further embodiments, the kit is configured for veterinary applications, detection in subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to detect gene rearrangements. For example, the instructions may include instructions to: provide a quantity of a polynucleotide that includes a region of interest; use the restriction enzyme to cleave the polynucleotide to create polynucleotide fragments, a portion of which are capable of binding to a probe; combine a quantity of the probe with said polynucleotide fragments to hybridize at least a portion of the polynucleotide fragments; remove a substantial quantity of the hybridized polynucleotide fragments to create a remaining sample and a removed sample; remove a substantial quantity of the hybridized polynucleotide fragments to create the remaining sample and the removed sample by providing a solid matrix comprising avidin or similar avidin-binding proteins and contacting a biotinylated polynucleotide fragment to the avidin, wherein the biotinylated polynucleotide fragment binds to the avidin and is removed from the sample to create the remaining sample and the removed sample; introduce to the remaining sample or the removed sample a quantity of a first primer pair targeting a first section of the polynucleotide and a quantity of a second primer pair targeting a second section of the polynucleotide, together or separately; amplify in a same reaction or in separate reactions the first and second sections to generate a first product and a second product; and/or quantitate the amount of the first product and the second product to detect the gene rearrangement by using a technique selected from the group consisting of quantitative PCR, gel electrophoresis, using microbeads in combination with flow cytometry, using gene-chip analysis, and using microarray analysis.

Optionally, the kit also contains other useful components, such as, test tubes, microbeads, biological microbeads, gene-chips, diluents, buffers, syringes, catheters, applicators, pipetting or measuring tools, or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit. The packaging material is constructed by well known methods, preferably to provide a sterile, contaminant-free environment. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### 5′/3′ Ratioing of the MLL Gene Rearrangement Modeled by EcoRV Cleaved Plasmid DNA

As a model for MLL gene rearrangement, the inventors used plasmid DNA (pMEPMLL) (accession number U04737), which comprise a human breakpoint cluster region of the MLL gene (SEQ ID NO. 1). Gene rearrangement at a bcr close to the 3′ end of the MLL locus was modeled by EcoRV restriction enzyme digestion. The DNA was digested with EcoRV which cleaves the DNA in the region that is common for translocations of the MLL gene. This model represents a rearranged gene. A hybridization step using a 3′ probe is performed to remove most or all of the template containing the B region (see FIG. 2A). Therefore, only or mostly the A region was copied during the two PCR reactions. One primer pair (SEQ ID NO.: 2 and SEQ ID NO.: 3) was designed to produce a 778 bp product (“A”) on the 5′ side of a putative breakpoint in the MLL bcr. Another primer pair (SEQ ID NO.: 4 and SEQ ID NO.: 5) was designed to produce a 541 bp product (“B”) on the 3′ side of the putative breakpoint.

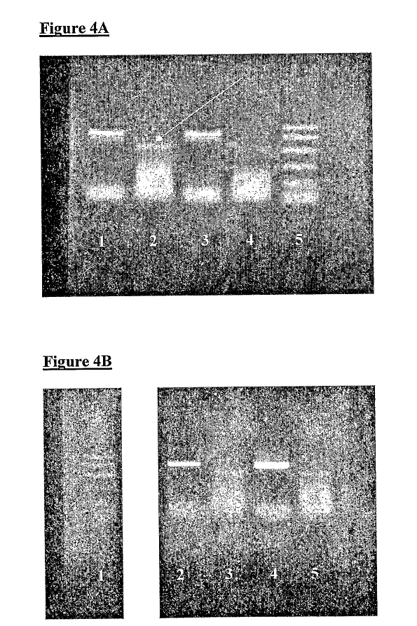

As shown by the arrow in FIG. 4, almost no product was produced in the PCR reaction for the B region. The small amount of the “B” product is the weak band; the smear below it was due to the reannealed primers. This indicates that in a clinical sample containing a translocation in the bcr close to the 3′ end of the MLL locus, similar results will be seen.

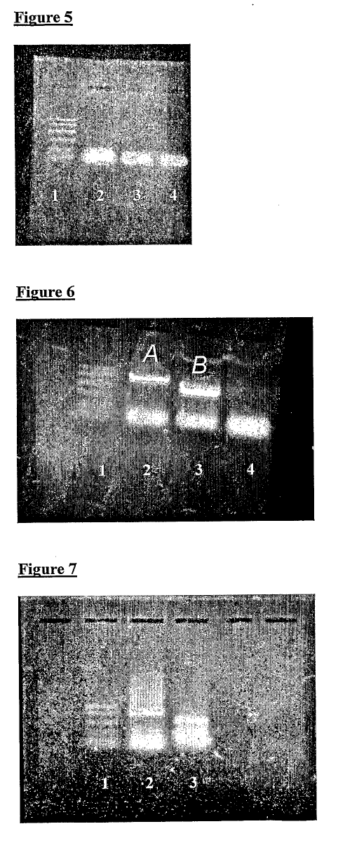

With intact, supercoiled plasmid DNA, which represents an unrearranged gene, no PCR products were detected up to 20 ng of template (see FIG. 5). When the DNA is intact (e.g., unrearranged), it will be completely or almost completely removed in the hybridization step. In the instance where complete removal of the DNA during the hybridization step is possible, no PCR products are detected. In the instance where almost complete removal of the DNA is perform thereby leaving a portion of the template DNA, two identical bands for A and B are detected.

As seen by EcoRV-cleaved plasmid DNA without the hybridization step, both products A and B are equally detected (FIG. 6).

### Example 2

### 5′/3′ Ratioing Modeled on Human Genomic DNA

Human genomic DNA with germline MLL was used as a model system. Human genomic DNA was digested with the BamH1 restriction enzyme to generate DNA fragments.

EcoRV was used to cleave human genomic DNA to create a model representing gene rearrangements. As seen in FIG. 7, the B product greatly decreased at a 2 μg of sample DNA, which indicates the operability of this inventive method. This difference diminished when the binding capacity of the immobilized DNA is saturated using 4-8-12 μg of sample DNA (data not shown), thus the hybridization and removal step is import to remove the 3′ fragments and the unrearranged genes (in embodiments where a 3′ probe is used).

### Example 3

### Detection of MLL Gene Rearrangement in Genomic DNA with Use of a 3′Probe

The translocation breakpoints of the MLL gene almost always fall within a region of 8.3 kilobases, referred to as the MLL breakpoint cluster region (bcr).

A sample comprising genomic DNA is digested with the restriction enzyme, BamHI. The use of BamHl generates numerous fragments of DNA, including a fragment comprising the MLL bcr. For purposes of clarity, the fragment of the MLL bcr is designated to have a 5′ fragment and a 3′ fragment. The 5′ fragment is the fragment of the MLL bcr that is on the 5′ side of a putative breakpoint at which a translocation may occur. The 3′ fragment is the fragment of the MLL bcr that is on the 3′ side of the putative breakpoint.

After digestion with BamHI, a hybridization step is performed with a 3′ probe. The 3′ probe may be immobilized to a solid support matrix. In such embodiments, the sample may be placed in contact with the support matrix containing the 3′ probe. The 3′ probe will hybridize with a portion the 3′ fragment of the MLL bcr and upon removal of the sample from the support matrix, the 3′ probe will remove the DNA molecules that hybridized with it. The hybridization and removal steps may be repeated two to three times to improve the removal process. Hybridization may also be performed in solution, followed by binding of the hybridized molecules containing biotin to the solid matrix. The DNA molecules removed may include the unrearranged MLL bcr (i.e., both the 5′ fragment and the 3′ fragment will be removed because the 5′ fragment and the 3′ fragment stay continuous as one fragment). The DNA molecules that are also removed include the 3′ fragment of a rearranged MLL bcr. The 3′ fragment of the rearranged MLL bcr may be an independent fragment, or it may have translocated onto another gene in which case the other gene is removed along with the 3′ fragment. If rearrangement of the MLL bcr is present, what remains in the sample after the hybridization and removal steps with the 3′ probe is the 5′ fragment of a rearranged MLL bcr. The hybridization and removal step may not be completely effective and thus, there may be some 3′ fragments that remain in the sample. Also remaining in the sample, although they may not be of any interest, are the other genes or DNA molecules with which the 3′ probes do not hybridize and thus are not removed.

Two primer pairs were designed. One pair (SEQ ID NO.: 6; SEQ ID NO.: 7) was designed to produce a 408 bp product (“A”; SEQ ID NO.: 8) on the 5′ side of a putative breakpoint in the MLL bcr. Another pair (SEQ ID NO.: 9; SEQ ID NO.: 10) was designed to produce a 573 bp product (“B”; SEQ ID NO.:11) on the 3′ side of the putative breakpoint.

After the hybridization and removal steps, the sample is quantitated by qPCR for the presence of A and B. PCR reactions using the primer pairs for product A and product B are performed. This may be performed in one reaction using all four primers (both pairs of primers). Alternatively, this may be performed in two separate reactions, using one pair of primers and a portion of the sample for a reaction to generate product A, and using the a pair of primers and another portion of the sample for a reaction to generate product B.

If rearrangements on the MLL bcr are present, a larger quantity of product A is generated and a smaller quantity of product B is generated. Some 3′ fragments may have remained in the sample for various reasons, for example, due to saturation of the 3′ probe. Thus, a small quantity of product B is generated. In a perfect system, if rearrangements of the MLL bcr are present, only product A is seen and no product B is seen.

In a perfect system, if no rearrangements on the MLL bcr are present, neither product A nor product B are generated. However, since some nonrearranged MLL bcr may have remained in the sample for various reasons, for example, due to the saturation of the 3′ probe, equal amounts of A and B are generated. The detection of the PCR products, A and/or B may be made on an agarose gel by electrophoresis.

### Example 4

### Detection of MLL Gene Rearrangement in Genomic DNA with Use of a 5′Probe

The present invention may also be performed using a 5′ probe and quantitating the presence of the 3′ fragment. In such embodiments, similar to example 3, a sample comprising genomic DNA is digested with the restriction enzyme, BamHI. The use of BamHI generates numerous fragments of DNA, including a fragment comprising the MLL bcr. For purposes of clarity, the fragment of the MLL bcr is designated to have a 5′ fragment and a 3′ fragment. The 5′ fragment is the fragment of the MLL bcr that is on the 5′ side of a putative breakpoint at which a translocation may occur. The 3′ fragment is the fragment of the MLL bcr that is on the 3′ side of the putative breakpoint.

After digestion with BamHI, a hybridization step is performed with a 5′ probe. The 5′ probe may be immobilized to a solid support matrix. In such embodiments, the sample may be placed in contact with the support matrix containing the 5′ probe. The 5′ probe will hybridize with a portion the 5′ fragment of the MLL bcr and upon removal of the sample from the support matrix, the 5′ probe will remove the DNA molecules that hybridized with it. The hybridization and removal steps may be repeated two to three times to improve the removal process. The DNA molecules removed may include the unrearranged MLL bcr (i.e., both the 5′ fragment and the 3′ fragment will be removed because the 5′ fragment and the 3′ fragment stay continuous as one fragment). The DNA molecules that are also removed include the 5′ fragment of a rearranged MLL bcr. The 5′ fragment of the rearranged MLL bcr may be an independent fragment, or it may have translocated onto another gene in which case the other gene is removed along with the 5′ fragment. If rearrangement of the MLL bcr is present, what remains in the sample after the hybridization and removal steps with the 5′ probe is the 3′ fragment of a rearranged MLL bcr. The hybridization and removal step may not be completely effective and thus, there may be some 5′ fragments that remain in the sample. Also remaining in the sample, although they may not be of any interest, are the other genes or DNA molecules with which the 5′ probes do not hybridize and thus are not removed.

Two primer pairs were designed. One pair (SEQ ID NO.: 6; SEQ ID NO.: 7) was designed to produce a 408 bp product (“A”; SEQ ID NO.: 8) on the 5′ side of a putative breakpoint in the MLL bcr. Another pair (SEQ ID NO.: 9; SEQ ID NO.: 10) was designed to produce a 573 bp product (“B”; SEQ ID NO.:11) on the 3′ side of the putative breakpoint.

After the hybridization and removal steps, the sample is quantitated by qPCR for the presence of A and B. PCRs using the primer pairs for product A and product B are performed. This may be performed in one reaction using all four primers (both pairs of primers). Alternatively, this may be performed in two separate reactions, using one pair of primers and a portion of the sample for a reaction to generate product A, and using the a pair of primers and another portion of the sample for a reaction to generate product B.

If rearrangements on the MLL bcr are present, a larger quantity of product B is generated and a smaller quantity of product A is generated. Some 5′ fragments may have remained in the sample for various reasons, for example, due to saturation of the 5′ probe. Thus, a small quantity of product A is generated. In a perfect system, if rearrangements of the MLL bcr are present, only product B is seen and no product A is seen.

In a perfect system, if no rearrangements on the MLL bcr are present, neither product A nor product B are generated. However, since some nonrearranged MLL bcr may have remained in the sample for various reasons, for example, due to the saturation of the 5′ probe, equal amounts of A and B are generated. The detection of the PCR products, A and/or B may be made on an agarose gel by electrophoresis.

### Example 5

### Detection of MLL Gene Rearrangement in cDNA with Use of a 3′Probe

The translocation breakpoints of the MLL gene almost always fall within a region of 8.3 kilobases, referred to as the MLL breakpoint cluster region (bcr).

A sample comprising the cDNA of the MLL gene is digested with the restriction enzyme, BamHI. The use of BamHI generates fragments of DNA, including a fragment comprising the MLL bcr. For purposes of clarity, the fragment of the MLL bcr is designated to have a 5′ fragment and a 3′ fragment. The 5′ fragment is the fragment of the MLL bcr that is on the 5′ side of a putative breakpoint at which a translocation may occur. The 3′ fragment is the fragment of the MLL bcr that is on the 3′ side of the putative breakpoint.

After digestion with BamHI, a hybridization step is performed with a 3′ probe. The 3′ probe may be immobilized to a solid support matrix. In such embodiments, the sample may be placed in contact with the support matrix containing the 3′ probe. The 3′ probe will hybridize with a portion the 3′ fragment of the MLL bcr and upon removal of the sample from the support matrix, the 3′ probe will remove the DNA molecules that hybridized with it. The hybridization and removal steps may be repeated two to three times to improve the removal process. The DNA molecules removed may include the unrearranged MLL bcr (i.e., both the 5′ fragment and the 3′ fragment will be removed because the 5′ fragment and the 3′ fragment stay continuous as one fragment). The DNA molecules that are also removed include the 3′ fragment of a rearranged MLL bcr. The 3′ fragment of the rearranged MLL bcr may be an independent fragment, or it may have translocated onto another gene in which case the other gene is removed along with the 3′ fragment. If rearrangement of the MLL bcr is present, what remains in the sample after the hybridization and removal steps with the 3′ probe is the 5′ fragment of a rearranged MLL bcr. The hybridization and removal step may not be completely effective and thus, there may be some 3′ fragments that remain in the sample. Also remaining in the sample, although they may not be of any interest, are the other genes or DNA molecules with which the 3′ probes do not hybridize and thus are not removed.

Two primer pairs were designed. One pair was designed to produce a 718 bp product (“A”) on the 5′ side of a putative breakpoint in the MLL bcr. Another pair was designed to produce a 541 bp product (“B”) on the 3′ side of the putative breakpoint.

After the hybridization and removal steps, the sample is quantitated by qPCR for the presence of A and B. PCRs using the primer pairs for product A and product B are performed. This may be performed in one reaction using all four primers (both pairs of primers). Alternatively, this may be performed in two separate reactions, using one pair of primers and a portion of the sample for a reaction to generate product A, and using the a pair of primers and another portion of the sample for a reaction to generate product B.

If rearrangements on the MLL bcr are present, a larger quantity of product A is generated and a smaller quantity of product B is generated. Some 3′ fragments may have remained in the sample for various reasons, for example, due to saturation of the 3′ probe. Thus, a small quantity of product B is generated. In a perfect system, if rearrangements of the MLL bcr are present, only product A is seen and no product B is seen.

In a perfect system, if no rearrangements on the MLL bcr are present, neither product A nor product B are generated. However, since some nonrearranged MLL bcr may have remained in the sample for various reasons, for example, due to the saturation of the 3′ probe, equal amounts of A and B are generated. The detection of the PCR products, A and/or B may be made on an agarose gel by electrophoresis.

### Example 6

### Use of Microarray Analysis

A sample from the above examples may be quantitiated by microarray analysis. In such embodiments, after the hybridization and removal steps are performed, a portion of the sample may be placed in contact with a microarray analysis chip.

In one alternative embodiment, after the hybridization and removal steps, PCRs using the primer pairs for product A and product B are performed. This may be performed in one reaction using all four primers (both pairs of primers). Alternatively, this may be performed in two separate reactions, using one pair of primers and a portion of the sample for a reaction to generate product A, and using the a pair of primers and another portion of the sample for a reaction to generate product B. After performing PCR, the sample or samples may be placed in contact with the microarray analysis chip.

The chip may be configured with two sets of oligonucleotide probes to detect the presence of the 5′ fragment and the presence of the 3′ fragment in the sample. One set of probes (e.g., oligonucleotide probes) can detect for the presence of 5′ fragments and one set of oligonucleotide probes can detect for the presence of 5′ fragments. If a 3′ probe was used to remove the 3′ fragments and the unrearranged gene or gene fragment, and the chip detect only the 5′ fragments or a higher amount of 5′ fragments as compared to the amount of 3′ fragments, it would indicate the presence gene rearrangements. If neither a 5′ fragment nor a 3′ fragment is detected on the chip or if substantially equal amounts of 5′ fragments and 3′ fragments are detected on the chip, it would indicate that no gene rearrangement is present.

In another alternative embodiment, after the hybridization and removal steps, all the remaining polynucleotides (e.g., genomic DNA, cDNA, mRNA) could be amplified by random amplification and used to hybridize the microarray.

While the description above refers to particular embodiments of the present invention, it should be readily apparent to people of ordinary skill in the art that a number of modifications may be made without departing from the spirit thereof. The accompanying claims are intended to cover such modifications as would fall within the true spirit and scope of the invention. The presently disclosed embodiments are, therefore, to be considered in all respects as illustrative and not restrictive, the scope of the invention being indicated by the appended claims rather than the foregoing description. All changes that come within the meaning of and range of equivalency of the claims are intended to be embraced therein.

What is claimed is:

1. A method of detecting a gene rearrangement, comprising:
providing a quantity of a polynucleotide, said polynucleotide including a region of interest;
cleaving said polynucleotide to create polynucleotide fragments, a portion of which are capable of binding to a probe;
combining a quantity of the probe with said polynucleotide fragments to hybridize at least a portion of the polynucleotide fragments;
removing a substantial quantity of the hybridized polynucleotide fragments to create a remaining sample and a removed sample; and
quantitating the amount of a first section of the region of interest and a second section of the region of interest in the remaining sample or the removed sample to detect the gene rearrangement.

2. The method of claim 1, wherein quantitating the amount of the first section and the second section comprises:
introducing to the remaining sample or the removed sample a quantity of a first primer pair targeting the first section and a quantity of a second primer pair targeting the second section;
amplifying the first and second sections to generate a first product and a second product; and
quantitating the amount of the first product and the second product to detect the gene rearrangement.

3. The method of claim 1, wherein said polynucleotide is selected from the group consisting of genomic DNA, cDNA, mRNA and combinations thereof.

4. The method of claim 1, wherein cleaving said polynucleotide comprises using a restriction enzyme to cleave said polynucleotide.

5. The method of claim 1, wherein said probe is selected from the group consisting of a probe that is immobilized on a solid matrix, a probe that has an affinity tag, and combinations thereof.

6. The method of claim 5, wherein the affinity tag is biotin and biotinylated polynucleotide fragments are generated upon hybridization of the affinity tag to the polynucleotide fragments.

7. The method of claim 6, wherein removing a substantial quantity of the hybridized polynucleotide fragments to create the remaining sample and the removed sample comprises:
providing a solid matrix comprising avidin; and
contacting the biotinylated polynucleotide fragments to the avidin, wherein the biotinylated polynucleotide fragments bind to the avidin and are removed from the sample to create the remaining sample and the removed sample.

8. The method of claim 2, wherein introducing to the remaining sample or the removed sample the quantity of the first primer pair and the quantity of the second primer pair comprises:
introducing to the remaining sample or the removed sample the quantity of the first primer pair and the quantity of the second primer pair together; or
introducing to the remaining sample or the removed sample the quantity of the first primer pair and the quantity of the second primer pair separately.

9. The method of claim 2, wherein amplifying the first and second sections to generate a first product and a second product comprises:
amplifying the first and second sections to generate a first product and a second product in a same reaction; or
amplifying the first and second sections to generate a first product and a second product in separate reactions.

10. The method of claim 1, wherein said region of interest is a mixed-lineage leukemia (MLL) breakpoint cluster region (bcr).

11. The method of claim 1, wherein said probe is a probe that targets the second section, and
wherein if the remaining sample comprises a larger quantity of the first section as compared to the second section, it indicates the presence of the gene rearrangement; if the remaining sample comprises a substantially equal quantity of the first section and the second section, it indicates a lack of the gene rearrangement; and if the remaining sample lacks the first section and the second section, it indicates a lack of the gene rearrangement; or
wherein if the removed sample comprises a larger quantity of the second section as compared to the first section, it indicates the presence of the gene rearrangement; and if the removed sample comprises a substantially equal quantity of the first section and the second section, it indicates a lack of the gene rearrangement.

12. The method of claim 1, wherein said probe is a probe that targets the first section, and
wherein if the remaining sample comprises a larger quantity of the second section as compared to the first section, it indicates the presence of the gene rearrangement; if the remaining sample comprises a substantially equal quantity of the first section and the second section, it indicates a lack of the gene rearrangement; and if the remaining sample lacks the first section and the second section, it indicates a lack of the gene rearrangement; or
wherein if the removed sample comprises a larger quantity of the first section as compared to the second section, it indicates the presence of the gene rearrangement; and if the removed sample comprises a substantially equal quantity of the first section and the second section, it indicates a lack of the gene rearrangement.

13. The method of claim 1, wherein quantitating the amount of the first section and the second section is performed by a technique selected from the group consisting of quantitative polymerase chain reaction (qPCR), gel electrophoresis, using microbeads in combination with flow cytometry, using gene-chip analysis, using microarray analysis and combinations thereof.

14. A method to diagnose a propensity to develop a disease condition, comprising:
providing a quantity of a polynucleotide, said polynucleotide including a region of interest;
cleaving said polynucleotide to create polynucleotide fragments, a portion of which are capable of binding to a probe;
combining a quantity of the probe with said polynucleotide fragments to hybridize at least a portion of the polynucleotide fragments;
removing a substantial quantity of the hybridized polynucleotide fragments to create a remaining sample and a removed sample;
quantitating the amount of a first section of the region of interest and a second section of the region of interest in the remaining sample or the removed sample to detect the gene rearrangement and diagnose the propensity to develop a disease condition.

15. The method of claim 1, wherein quantitating the amount of the first section and the second section comprises:
introducing to the remaining sample or the removed sample a quantity of a first primer pair targeting the first section and a quantity of a second primer pair targeting the second section;
amplifying the first and second sections to generate a first product and a second product; and
quantitating the amount of the first product and the second product to detect the gene rearrangement.

16. The method of claim 14, wherein the disease condition is cancer.

17. The method of claim 14, wherein said polynucleotide is selected from the group consisting of genomic DNA, cDNA, mRNA and combinations thereof.

18. The method of claim 14, wherein cleaving said polynucleotide comprises using a restriction enzyme to cleave said polynucleotide.

19. The method of claim 14, wherein said probe is selected from the group consisting of a probe that is immobilized on a solid matrix, a probe that has an affinity tag, and combinations thereof.

20. The method of claim 19, wherein the affinity tag is biotin and biotinylated polynucleotide fragments are generated upon hybridization of the affinity tag to the polynucleotide fragments.

21. The method of claim 20, wherein removing a substantial quantity of the hybridized polynucleotide fragments to create the remaining sample and the removed sample comprises:
providing a solid matrix comprising avidin; and
contacting the biotinylated polynucleotide fragments to the avidin, wherein the biotinylated polynucleotide fragments bind to the avidin and are removed from the sample to create the remaining sample and the removed sample.

22. The method of claim 15, wherein introducing to the remaining sample or the removed sample the quantity of the first primer pair and the quantity of the second primer pair comprises:
introducing to the remaining sample or the removed sample the quantity of the first primer pair and the quantity of the second primer pair together; or
introducing to the remaining sample or the removed sample the quantity of the first primer pair and the quantity of the second primer pair separately.

23. The method of claim 15, wherein amplifying the first and second sections to generate a first product and a second product comprises:
amplifying the first and second sections to generate a first product and a second product in a same reaction; or
amplifying the first and second sections to generate a first product and a second product in separate reactions.

24. The method of claim 14, wherein said region of interest is a mixed-lineage leukemia (MLL) breakpoint cluster region (bcr).

25. The method of claim 14, wherein said probe is a probe that targets the second section, and
wherein if the remaining sample comprises a larger quantity of the first section as compared to the second section, it indicates the presence of the gene rearrangement; if the remaining sample comprises a substantially equal quantity of the first section and the second section, it indicates a lack of the gene rearrangement; and if the remaining sample lacks the first section and the second section, it indicates a lack of the gene rearrangement; or
wherein if the removed sample comprises a larger quantity of the second section as compared to the first section, it indicates the presence of the gene rearrangement; and if the removed sample comprises a substantially equal quantity of the first section and the second section, it indicates a lack of the gene rearrangement.

26. The method of claim 14, wherein said probe is a probe that targets the first section, and
wherein if the remaining sample comprises a larger quantity of the second section as compared to the first section, it indicates the presence of the gene rearrangement; if the remaining sample comprises a substantially equal quantity of the first section and the second section, it indicates a lack of the gene rearrangement; and if the remaining sample lacks the first section and the second section, it indicates a lack of the gene rearrangement; or
wherein if the removed sample comprises a larger quantity of the first section as compared to the second section, it indicates the presence of the gene rearrangement; and if the removed sample comprises a substantially equal quantity of the first section and the second section, it indicates a lack of the gene rearrangement.

27. The method of claim 14, wherein quantitating the amount of the first section and the second section is performed by a technique selected from the group consisting of quantitative polymerase chain reaction (qPCR), gel electrophoresis, using microbeads in combination with flow cytometry, using gene-chip analysis, using microarray analysis and combinations thereof.

28. A kit for detecting a gene rearrangement, comprising:
a restriction enzyme;
a probe; and
instructions to use the restriction enzyme and the probe to detect the gene rearrangement.

29. The kit of claim 28, wherein the instructions comprise:
instructions to provide a quantity of a polynucleotide, said polynucleotide including a region of interest;
instructions to use the restriction enzyme to cleave said polynucleotide to create polynucleotide fragments, a portion of which are capable of binding to the probe;
instructions to combine a quantity of the probe with said polynucleotide fragments to hybridize at least a portion of the polynucleotide fragments;
instructions to remove a substantial quantity of the hybridized polynucleotide fragments to create a remaining sample and a removed sample;
instructions to quantitate the amount of a first section of the region of interest and a second section of the region of interest in the remaining sample or the removed sample to detect the gene rearrangement.

30. The kit of claim 29, wherein the instructions to quantitate the amount of the first section and the section comprise:
instructions to introduce to the remaining sample or the removed sample a quantity of a first primer pair targeting the first section and a quantity of a second primer pair targeting the second section;
instructions to amplify the first and second sections to generate a first product and a second product; and
instructions to quantitate the amount of the first product and the second product to detect the gene rearrangement.

31. The kit of claim 29, wherein said polynucleotide is selected from the group consisting of genomic DNA, cDNA, mRNA and combinations thereof.

32. The kit of claim 28, wherein said probe is selected from the group consisting of a probe that is immobilized on a solid matrix, a probe that has an affinity tag and combinations thereof.

33. The kit of claim 32, wherein the affinity tag is biotin and biotinylated polynucleotide fragments are generated upon hybridization of the affinity tag to the polynucleotide fragments.

34. The kit of claim 29, wherein instructions to remove a substantial quantity of the hybridized polynucleotide fragments to create the remaining sample and the removed sample comprises:
instructions to provide a solid matrix comprising avidin; and
instructions to contacting the biotinylated polynucleotide fragments to the avidin, wherein the biotinylated polynucleotide fragments bind to the avidin and are removed from the sample to create the remaining sample and the removed sample.

35. The kit of claim 30, wherein instructions to introduce to the remaining sample or the removed sample the quantity of the first primer pair and the quantity of the second primer pair comprises:
instructions to introduce to the remaining sample or the removed sample the quantity of the first primer pair and the quantity of the second primer pair together; or
instructions to introduce to the remaining sample or the removed sample the quantity of the first primer pair and the quantity of the second primer pair separately.

36. The kit of claim 30, wherein instructions to amplify the first and second sections to generate a first product and a second product comprises:
instructions to amplify the first and second sections to generate a first product and a second product in a same reaction; or
instructions to amplify the first and second sections to generate a first product and a second product in separate reactions.

37. The kit of claim 28, wherein the gene rearrangement is a gene rearrangement in a mixed-lineage leukemia (MLL) breakpoint cluster region (bcr).

38. The kit of claim 29, wherein the probe is a probe that targets the second section, and
wherein if the remaining sample comprises a larger quantity of the first section as compared to the second section, it indicates the presence of the gene rearrangement; if the remaining sample comprises a substantially equal quantity of the first section and the second section, it indicates a lack of the gene rearrangement; and if the remaining sample lacks the first section and the second section, it indicates a lack of the gene rearrangement; or
wherein if the removed sample comprises a larger quantity of the second section as compared to the first section, it indicates the presence of the gene rearrangement; and if the removed sample comprises a substantially equal quantity of the first section and the second section, it indicates a lack of the gene rearrangement.

39. The kit of claim 29, the probe is a probe that targets the first section of the polynucleotide, and
wherein if the remaining sample comprises a larger quantity of the second section as compared to the first section, it corroborates with a gene rearrangement, if the remaining sample comprises a substantially equal quantity of the first section and the second section, it corroborates with a lack of the gene rearrangement, and if the remaining sample lacks the first section and the second section corroborates with a lack of the gene rearrangement; or
wherein if the removed sample comprises a larger quantity of the first section as compared to the second section, it indicates the presence of the gene rearrangement; and if the removed sample comprises a substantially equal quantity of the first section and the second section, it indicates a lack of the gene rearrangement.

40. The kit of claim 29, wherein instructions to quantitate the amount of the first product and the second product comprise:
instructions to use a technique selected from the group consisting of quantitative polymerase chain reaction (qPCR), gel electrophoresis, using microbeads in combination with flow cytometry, using gene-chip analysis, using microarray analysis and combinations thereof.


SEQUENCE LISTING (11 sequences)

SEQ ID NO: 1 - DNA, 8342 bp, Homo Sapiens
ggatcctgccccaaagaaaagcagtagtgagcctcctccacgaaagcccgtcgaggaaaagagtgaagaagggaatgtctcggcccctgggcctgaatccaaacaggccaccactccagcttccaggaagtcaagcaagcaggtctcccagccagcactggtcatcccgcctcagccacctactacaggaccgccaagaaaagaagttcccaaaaccactcctagtgagcccaagaaaaagcagcctccaccaccagaatcaggtgagtgaggagggcaagaaggaattgctgacccacaagtactaacaaaaaagcactgatgtctcaaacagcatttgaaagcaggaaatgtatgatttgaagtcttcagttcaagaaaatcagctctctttctaactattatgtttaataataaagaaacagaaacaaaaaaaacagttaaattggaggtattgttttaatttcctgttcgaagcctagagtttaaatagttttttttttttttttctaatggccctttcttcacaggtcagtcagtactaaagtagtcgttgccagcatctgactgcaatttattctgaattttttaggtccagagcagagcaaacagaaaaaagtggctccccgcccaagtatccctgtaaaacaaaaaccaaaagaaaaggtgaggagagatttgtttctctgccatttctcagggatgtattctattttgtagggaaaagccttatccttgacttctatgtagatggcagtggaatttcttaaaattaagaaacttcaagtttaggcttttagctgggcacggtggctcacgctggtaatcccaacacttagtgaggctgaggtgggaggattgcttgaggccagcagttcaagaccagcctgggcaacatagcaagaccctgtctttatttaaaccaaaaaaaaaaaaagaagaagaagaagttagccaggcatggtggcagttgcgtgtagtcccaggtactcaggaggctgagatagaaggattgtcttgagcccaggaattcaaggctgtagtgagctatgattgtaccactgcagtccagcctgggtgacaaagcaaaacactgtctccaaaaaaaatttaggcttggcaaggcgcagcggctcacgcctgtgatcccagcactttgggaagccgaagcaggcagatcacttgaggtcaggagttggagaccagcctggccaacatggtgaaaccctgtctctactgaaaatacaaaaattagccggttgtggtagtgggtgcttggtaatcctagctacttgggaggctgaggcagggggaattgcctgaaacctgcgaggcggaggctgcagtgagccgagattgcatcattgcactctagcctggacaacagagctagactccatcccaaaaaaaaaaaaaaaaagtagccgggcacggtggctcacgcctgtaatcccagcactttgggaggccgaggcgggcggatcatgagggcaggagatcgagaccatcctggctaacacggtgaaaccctgtctctactaaaaatacaaaaaattagcccggcgaggtggcgggcgcctgtagtcccagctactcaggagagtgagccaggagaatggcgtgaacccggggggcggagcctgcagtgagccgagatcgcgccactgcactccagcttgggtgacaccgagactccgtctcaaaaaaaaataaaaagtttaggctttagcctgtttcttttttggtttcttccttgttgcttttcccttctttgtggccccacatgttctagcctaggaatctgcttattctaaaggccatttggcgtaattattttttgaccccaacatcctttagcaattatttgtctgtaaaaatcacccttccctgtattcactatttttatttattatggataaagagatagtgtggtggctcacatctataatcccagcactttgggggcccaaggcgggaggatcacttgagggcaggagctggagaccagcctgggcagcacagtgacacacagttgctataaaaaatttaaaacccaactaggcatggtggcatgcacctgtagtcccagctactcttgagaagctgaggcaggaggatcacgagcccacaaggtctaggctgcagtgagctgtgactgtgccactgtattgcagcctaggcaacaaagcaagacccagtctcttttaaaaaaaaattcaaagattattgtttatgttggaaacatgttttttagatctattaataaaatttgtcatttgcattattatctgttgcaaatgtgaaggcaaatagggtgtgattttgttctatattcatcttttgtctccttaggaaaaaccacctccggtcaataagcaggagaatgcaggcactttgaacatcctcagcactctctccaatggcaatagttctaagcaaaaaattccagcagatggagtccacaggatcagagtggactttaaggtaaaggtgttcagtgatcataaagtatattgagtgtcaaagactttaaataaagaaaatgctactaccaaaggtgttgaaagaggaaatcagcaccaactgggggaatgaataagaactcccattagcaggtgggtttagcgctgggagagctttggacagtgttgttaggtcactgtttgtgaactgactgcagaacatacataatgaaacattcctatccatcctgaggagtatcagaggaagtaattccttcacatggaaagtatcaaaccatgatgattccttgagtcagcaaaactgtaagagaaattcaatcccagtgtattttcgcaatatcttcactatgaattgaacaactaggtgagccttttaatagtccgtgtctgagattaaaactttttaaagcagcagttatttttggactcattgaaatgaaatactctgacattgtgatgtcacactaattttatgcttttcatccttattttccatccaaagttgtgtaattgtaaaactttcctaagtgacctttctctctccacaggaggattgtgaagcagaaaatgtgtgggagatgggaggcttaggaatcttgacttctgttcctataacacccagggtggtttgctttctctgtgccagtagtgggcatgtagaggtaaggcatcctgcttctttgtaccccaggaagtacataaatgattgatctggggatgagattactatagtctgttttgttggtatttagcaggtactattccctgtttaaaccagctaaagaaatgttttgaagtattttagagattttaggaaggaatctgctattagagtagcaaagttattgagagtgaaaagatcaataatcccatctctcttaaattcagtctttattagagttctgatctttctgttagatgtctaaataagagaaaaaattatacagtggtctattaaaagggatgctattgatggttattttatattgtatatcaaagcctcttcatctataaggagctcttaccaattaataagaaaaaggaatgacatccagaaaaaaaaataggcaaaagacagaaatagataattcacaaaattagaaataaatacatgttgggtggcagggggaggtgaagggagggtgtctgttttttagccctctagtgaccaaaaactggaaattaaagcatgataaaaaaagaatcctgaataaatggggactttctgttggtggaaagaaatatagattagttacaatctttctttctgagggaattatttggaaatatatatatctatctttaaaataggtatatcctctaacatagcaattgcacttcaaacacttatggatataattagataaattggcaaatctgtagatataaagaagtgttcatttcaatattgctcataataataaaaaactggaaacaacccgaaagtccatctatagggagcatgggttaaaataagcatagggcatatagctgggcacggtggctcacgcctgtaatcccagcactttgggaggccaaggcaggcggatcacaaggtcaggagatccagaccatcctggctaacacagtgaaaccccgtctctattaaaaatacaaaaaaattagccgggtgtggtggcgggcgcctgtagtcccagctactcgagaggctgaggcaggagaacggcatgaacccgggaggtggagcttgcagtgagccgagatcgccccactgcactcccgcctgggcgacagagcaagactccgtctcaaaaaaaaataaaagtgtagggcatatataatgccaaatatgaagtcctaaagataatatatattaatattattaggttggtccaaaagtaattgcagtaataacatggaaagatgtccatgacatatcactgagtgaaaagagcaggttacaagataatatataaagcacaatcccatcttagtttggaaaagtgtttttaaagtatatatctagaaaacaatctggaaggattcacaccaaaatattaagagtgtggttggattatgggtgacctttatttgtttctctggtttttttttttttaatctttctgagttttttggagtatgtaccacctttacaatgaggaaggaaaaagtagcacaattttaaataggaagcagtagtttgtcatttataagggacatatcctacatcctttacagttcttaaattcctggcagatacctctttggcttattacttaccacataagatatgtattcaaaggtggtaaagaaaatccacgtcgggtgcagtggctcacgcctgtaatcccagtactttgggaggctgacgcaggaggaccgcttgagctcaggagttcaagaccagcctgagcaccatagtgagacctcatctctactaaaaaaaaaataaaataccaggcatggtagcatgtgcctgtagtcccagctactctagtcccagctacttgggaggctgaggtgagaggatcacttgagcccaggagatcgaggctgcagtgagccattatcacgccactgcactccagcctgggcaactaagcaagaccctgtctcaaaaaaattttaaaaaatttaaaaaataagaaaatccaagctaggttgaaatctgaatgttgagcagtcagtgagacacaaactagctaagaaagtcaaccctgcccacttgccatttgaagttattactagcaaaattacaaattattgcctactattcatttactaagcaaatattctcttagtccctattacgaacaacttattgttctaagtgcagaagttcagatatcattgagactgagaatattcagtctacaagtgccaggggtctactgtatcctcttttccgtcttaatacagtgctttgcacccatatatatgccacccacaggaataactttttttatagcaccagtccttcaacttctgggattaaacagattttttttcagggtataattgttctgatctaaattctttatagttgtacatagcaatctcacagggttcctaaaatataaaatagagaatagcatgctgcctgcactgcactcctaaagcatgaccagtgcttgataaactctcctccatgcgaattttttaaactttttatgttgacatgatttcagacttacaaaaaaactatgagttgtacagagaattctaagtacccctcacccaaattccctaagtgttaatatgtttctctgtgtgtatatattttacaaaataacaaataaaatacatatacacattttacctgtagatacacatgtatctaaaaatttgagaacaagttggagacataaaccattttacctctaaatattttagtgtatatttttaaaaatcaaggacgttctcgtatttaaccatggtataattaccaaatcaggaaattaacacactgggacattactattatctgatctataggccttatttaggtttgaccaattgtcccaataattcctttatggcaaaagaaaattctggattatcctagttagtatttttgaaaatcctatatcaatatgaaaataacttatttctaaaattagaaatggaggctgggcgtggtggctcacgcctataatcccagcactttgggaggccgaggcaggcagatcacaaggtcaggagattgagaccatcctcgctaacacagtgaaaccccatctctactaaaaatacaaaaaattagccaggtgtggtgggacgcgcctgtgatcccagctactcaggagactgaggctggagaatcgcttgaacccaacaggcggagggttgcagtgagtcgagatcgcaccactgcaccccagcctgggcgacagcgagactccgtctcaaaaaaataaataaataaaaattaaaacaattaaaaaaataaaattacaaatggaaaggacaaaccagaccttacaactgtttcgtatattacagaaaacgtttaaaccctccctatttcccccaccccactcctttatattcccatagctctttgtttataccactcttaggtcacttagcatgttctgttaaatcttgtattatatttattttgttactttctatttccactggtattaccactttagtactctgaatctcccgcaatgtccaatactgtacttttttacatagtcattgcttaatgaatatgtattgaattaaatatatgccagtggactactaaaacccaaagtatataagaagggtatggttgattatgtttttctacatattatttgacatacttctatcttcccatgttcttactatagtttgtgtattgccaagtctgttgtgagcccttccacaagttttgtttagaggagaacgagcgccctctggaggaccagctggaaaattggtgttgtcgtcgctgcaaattctgtcacgtttgtggagggcaacatcaggctacaaaggtacaaaacttggtaatagaactacagctgggcctctgtatcagtgggttctgtatccctggactcaaccaaccttggattgaatgtatctgggaaaaaatgagtagttgcctctgtactctatgtgaacagactttttcttgtcattatttcctaaacaatacagtataacaactatttacattgtattaggtatgataagtaatctagagataatttaaagtatatggtgggcggatcacttgaagccaggagttcgagaccagcctgagccaacatggtgaaaccccatctctactaaaaatacaaaaaattagccaggtgtggtggtgggcacctgtagtcccagctacttgggaggctgagggaggaaaatcgcttgaactttggaggcagaggttgcagtgagccactccagcctgtggtgcagtctgtcactccagcctgggtgacacagtgagactccatctcaaaaaaaaaaaaaaaaaaaaaactatatgggaggatgtgcattttgttatatgcaaatgctgcaccattttgtctagggacttgggcatccatggactttggtatcctctgggggtcctggaaccaatcccccatggaaaccaaggatgactgtgcttagagtattgctttctttcttgatttgtatttctgtcttccagttaagattttgtatctatattatttctctttttacttagtctgtctttagcatttaattgggtgtaatcagttgcctattttgtgttttaattttgggactatagcagaaaacatgatgttgaataaaattccaaaaataagtcaaatctacctaatatgaatactcatcactgagtgcctttggcaggaaataaatctatctcaatgcgttaattgggagtaaataatgcatgaggaaatttaaactcataattgtgtgctgtacttacttgccagtaaatgtgaaatggggtactaagtaataggtgttgggtgaaggtaatatgatgcttatctttttgccattatattttcttacagcagctgctggagtgtaataagtgccgaaacagctatcaccctgagtgcctgggaccaaactaccccaccaaacccacaaagaagaagaaagtctgggtgagttatacacatgatgctcttttatagagaaccaccatgtgactattggacttatgtaacttgtattacaaatatctatgcatgaggatgtcagtatgacaatctttttccctcattactaggaaatcatctcaggagagaaattaaatctataaatggatgcatttaagatctttttagttaagtaaagatattaaaaacaagaaattcctattgaatttcttttcttcttttctagatctgtaccaagtgtgttcgctgtaagagctgtggatcc

SEQ ID NO: 2 - DNA, 23 bp, Artificial Sequence
  Primer
ggtcaataagcaggagaatgcag

SEQ ID NO: 3 - DNA, 23 bp, Artificial Sequence
  Primer
tctacatgcccactactggcaca

SEQ ID NO: 4 - DNA, 24 bp, Artificial Sequence
  Primer
gtgtattgccaagtctgttgtgag

SEQ ID NO: 5 - DNA, 24 bp, Artificial Sequence
  Primer
tctgcctccaaagttcaagcgatt

SEQ ID NO: 6 - DNA, 29 bp, Artificial Sequence
  Primer
gcagcctccaccaccagaatcaggtgagt

SEQ ID NO: 7 - DNA, 27 bp, Artificial Sequence
  Primer
gttttacagggatacttgggcggggag

SEQ ID NO: 8 - DNA, 409 bp, Homo Sapiens
gcagcctccaccaccagaatcaggtgagtgaggagggcaagaaggaattgctgacccacaagtactaacaaaaaagcactgatgtctcaaacagcatttgaaagcaggaaatgtatgatttgaagtcttcagttcaagaaaatcagctctctttctaactattatgtttaataataaagaaacagaaacaaaaaaaacagttaaattggaggtattgttttaatttcctgttcgaagcctagagtttaaatagttttttttttttttttctaatggccctttcttcacaggtcagtcagtactaaagtagtcgttgccagcatctgactgcaatttattctgaattttttaggtccagagcagagcaaacagaaaaaagtggctccccgcccaagtatccctgtaaaac

SEQ ID NO: 9 - DNA, 31 bp, Artificial Sequence
  Primer
atatgaatactcatcactgagtgcctttggc

SEQ ID NO: 10 - DNA, 32 bp, Artificial Sequence
  Primer
agctcttacagcgaacacacttggtacagatc

SEQ ID NO: 11 - DNA, 574 bp, Homo Sapiens
atatgaatactcatcactgagtgcctttggcaggaaataaatctatctcaatgcgttaattgggagtaaataatgcatgaggaaatttaaactcataattgtgtgctgtacttacttgccagtaaatgtgaaatggggtactaagtaataggtgttgggtgaaggtaatatgatgcttatctttttgccattatattttcttacagcagctgctggagtgtaataagtgccgaaacagctatcaccctgagtgcctgggaccaaactaccccaccaaacccacaaagaagaagaaagtctgggtgagttatacacatgatgctcttttatagagaaccaccatgtgactattggacttatgtaacttgtattacaaatatctatgcatgaggatgtcagtatgacaatctttttccctcattactaggaaatcatctcaggagagaaattaaatctataaatggatgcatttaagatctttttagttaagtaaagatattaaaaacaagaaattcctattgaatttcttttcttcttttctagatctgtaccaagtgtgttcgctgtaagagct